(19) United States
(12) Patent Application Publication
(10) Pub. No.: US 2025/0115858 A1
(43) Pub. Date: Apr. 10, 2025

(54) CULTURE APPARATUS AND METHOD

(71) Applicants: Canon Kabushiki Kaisha, Tokyo (JP); CANON MEDICAL SYSTEMS CORPORATION, Tochigi (JP)

(72) Inventors: Noritaka UKIYO, Tokyo (JP); Yosuke YAMAGUCHI, Kawasaki (JP); Hirotoshi TAHARA, Nasushiobara (JP)

(73) Assignees: Canon Kabushiki Kaisha, Tokyo (JP); CANON MEDICAL SYSTEMS CORPORATION, Tochigi (JP)

(21) Appl. No.: 18/906,387

(22) Filed: Oct. 4, 2024

(30) Foreign Application Priority Data
Oct. 6, 2023 (JP) 2023-174555

(51) Int. Cl. C12M 1/34; C12M 1/00

(52) U.S. Cl. CPC C12M 41/34; C12M 23/34; C12M 29/06; C12M 29/20

(57) ABSTRACT

A culture apparatus according to embodiments includes a gas supplier, a culture chamber, a first flow path, and a gas suction equipment. The gas supplier is configured to supply gas. The culture chamber is configured to culture a culture. The first flow path is configured to allow gas supplied by the gas supplier to flow to the culture chamber. The gas suction equipment is configured to allow gas in the culture chamber to be discharged to the outside through a second flow path.

### CROSS-REFERENCE TO RELATED APPLICATIONS

This application is based upon and claims the benefit of priority from Japanese Patent Application No. 2023-174555, filed on Oct. 6, 2023; the entire contents of which are incorporated herein by reference.

### FIELD

Embodiments disclosed herein relate to a culture apparatus and a method.

### BACKGROUND

Conventionally, cell culture is conducted in an environment set to be most suitable for cells to be cultured. For example, in the culture of induced pluripotent stem cells (iPS cells), culture is conducted in a culture chamber controlled to “temperature: 37° C., humidity: 90%, and CO₂ concentration: 5%” in order to maintain a culture medium environment.

For example, a method that supplies water vapor into a culture chamber by a supply device provided outside the culture chamber is known as a means for controlling the environment in the culture chamber.

However, the conventional method that supplies water vapor into a culture chamber by a supply device provided outside the culture chamber sometimes fails to introduce an atmosphere containing water vapor at a desired concentration into the culture chamber.

### BRIEF DESCRIPTION OF THE DRAWINGS

FIG. 1 is an external view of a culture apparatus according to a first embodiment;

FIG. 2 is a diagram illustrating an incubator according to the first embodiment;

FIG. 3 is a diagram illustrating a cartridge according to the first embodiment;

FIG. 4A is a schematic diagram illustrating an exemplary configuration of the culture apparatus according to the first embodiment;

FIG. 4B is a schematic diagram illustrating an exemplary configuration of the culture apparatus according to the first embodiment;

FIG. 5A is a schematic diagram illustrating an exemplary configuration of the culture apparatus according to a modification; and

FIG. 5B is a schematic diagram illustrating an exemplary configuration of the culture apparatus according to the modification.

### DETAILED DESCRIPTION

A culture apparatus according to embodiments includes a gas supplier, a culture chamber, a first flow path, and a gas suction equipment. The gas supplier is configured to supply gas. The culture chamber is configured to culture a culture. The first flow path is configured to allow gas supplied by the gas supplier to flow to the culture chamber. The gas suction equipment is configured to allow gas in the culture chamber to be discharged to the outside through a second flow path.

Embodiments of a culture apparatus and a method according to the present application will be described in detail below with reference to the accompanying drawings. It should be noted that the culture apparatus and the method according to the present application are not limited by the embodiments described below. In the following, a case where a culture chamber for culturing a culture is provided in a cartridge installed in an incubator in which a culture environment is formed will be described by way of example. In the following description, similar constitutional elements are denoted by common reference signs and an overlapping description is omitted.

### First Embodiment

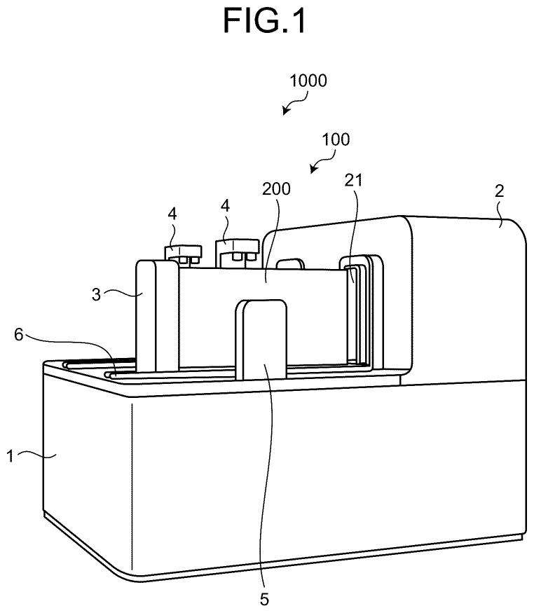

FIG. 1 is an external view of a culture apparatus 1000 according to a first embodiment. As illustrated in FIG. 1, the culture apparatus 1000 according to the present embodiment includes an apparatus body 100 including an apparatus base 1, an incubator 2, a cooling mechanism 3, fluid feeding mechanisms 4, a valve opening/closing mechanism 5, and a transfer mechanism 6, and a cartridge 200. Culture is performed using the cartridge 200.

The cartridge 200 has a culture vessel for culturing a culture (e.g., cells) to be cultured and is installed in the incubator 2 of the apparatus body 100. The cartridge 200 also includes a reagent, a closed-system flow path, and the like, and is configured to perform various processes related to cell culture. The cartridge 200 is an example of the vessel.

The apparatus base 1 holds the incubator 2, the cooling mechanism 3, the fluid feeding mechanisms 4, the valve opening/closing mechanism 5, and the transfer mechanism 6, and controls each of the components. For example, the apparatus base 1 includes processing circuitry for controlling each of the components as described above, and the processing circuitry executes control on each of the components described below, thereby controlling the entire apparatus body 100.

The incubator 2 has a gas supplier to supply gas, a temperature controller to control temperatures, various sensors, and the like, and controls the interior space formed inside to achieve an environment suitable for culture. Specifically, the incubator 2 controls the temperature, humidity, CO₂ concentration, and the like in the interior space to attain a substantially constant condition. For example, the incubator 2 controls the environment in the interior space to attain “temperature: 37° C., humidity: 90%, and CO₂ concentration: 5%”.

Here, as illustrated in FIG. 1, the incubator 2 has an opening 21 that communicatively connects the inside and outside of the interior space, and the cartridge 200 is installed in the interior space through the opening 21. The incubator 2 is an example of an enclosure.

The cooling mechanism 3 cools a reagent held in the cartridge 200.

The fluid feeding mechanisms 4 each control feeding of fluid in a flow path provided in the cartridge 200. Specifically, the fluid feeding mechanism 4 is a pressurizing mechanism that applies energy for feeding a cellular fluid containing cells or a reagent to a flow path in the cartridge 200. For example, the fluid feeding mechanism 4 is connected to a connection port on the top surface of the cartridge 200 to apply energy to the inside of the cartridge, thereby feeding a cellular fluid or a reagent to a flow path in the cartridge 200.

The valve opening/closing mechanism 5 controls a switching valve for switching flow paths in the cartridge 200. Specifically, in each process of cell culture in the cartridge 200, the valve opening/closing mechanism 5 controls the opening and closing of a switching valve in a closed-system flow path to switch flow paths in the cartridge 200, and allows a reagent, a cell fluid, or the like for use in each process to flow in the flow path.

The transfer mechanism 6 transfers the cartridge 200 through the opening 21 into the interior space of the incubator 2. Specifically, the transfer mechanism 6 has a drive source such as a motor and a support mechanism that supports the cartridge 200, and transmits drive force generated by the drive source to the support mechanism to move the cartridge 200 linearly. For example, the transfer mechanism 6 moves the cartridge 200 toward the incubator 2 to install the cartridge 200 in the incubator and moves the cartridge 200 out of the incubator 2 to eject the cartridge 200 from the inside of the incubator 2.

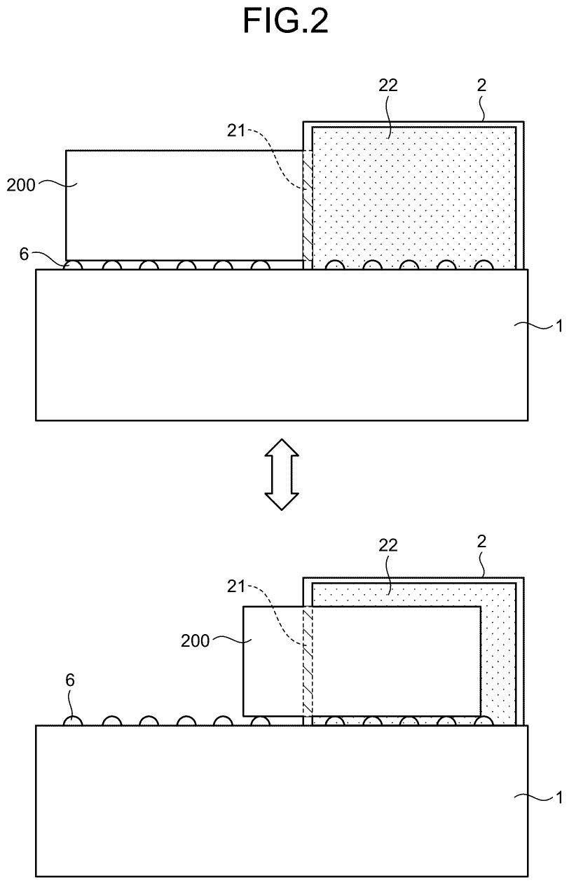

Here, in the culture apparatus 1000 according to the present embodiment, a part of the cartridge 200 is installed in the interior space of the incubator 2. FIG. 2 is a diagram illustrating the incubator 2 according to the first embodiment. FIG. 2 schematically illustrates the culture apparatus 1000 viewed from a direction orthogonal to the direction of movement of the cartridge 200 by the transfer mechanism 6.

As illustrated in the upper diagram in FIG. 2, the incubator 2 has an interior space 22 inside, and has the opening 21 on the cartridge 200 side. The incubator 2 has the gas supplier, the temperature controller, various sensors, and the like to control the atmosphere in the interior space 22 to attain a condition suitable for cell culture (e.g., “temperature: 37° C., humidity: 90%, and CO₂ concentration: 5%”).

At least a part of the incubator 2, including the culture vessel in the cartridge 200 having the culture vessel, is installed in the interior space 22. In other words, in a process of cell culture, the cartridge 200 is partially inserted into the interior space 22 in the incubator 2, as illustrated in the lower diagram in FIG. 2. Here, the culture vessel is provided in a portion of the cartridge 200 that is installed in the interior space 22.

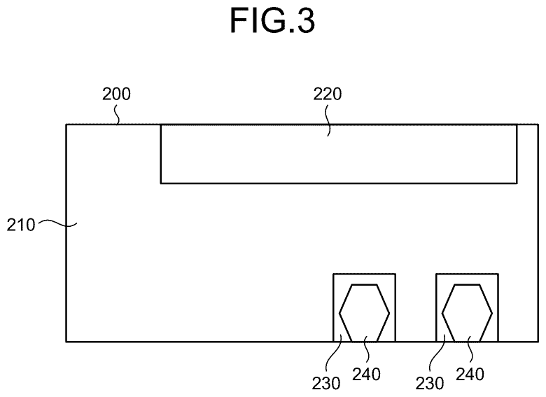

FIG. 3 is a diagram illustrating the cartridge 200 according to the first embodiment. FIG. 3 schematically illustrates a partial configuration in the inside of the cartridge 200. As illustrated in FIG. 3, the cartridge 200 is divided into a plurality of spaces inside and includes various components. For example, as illustrated in FIG. 3, the cartridge 200 has a space 210 in which a reagent or the like is provided, a space 220 in which a vessel for storing a cellular fluid or a reagent to be fed by the fluid feeding mechanism 4 is provided, spaces 230 in which culture vessels 240 are provided, and the like. The spaces are connected by flow paths so that each process related to cell culture is performed inside the cartridge 200. The example of the cartridge illustrated in FIG. 3 is only an example, and the spaces inside the cartridge can be set as desired. The spaces 230 are also an example of the culture chamber.

For example, when iPS cells are cultured, separation of nucleated cells from a liquid (e.g., blood) containing nucleated cells, expansion culture of CD34-positive cells, introduction of reprogramming factors, culture after introduction, and the like are carried out in the cartridge 200. When each of these processes is carried out, the cartridge 200 switches between the state illustrated in the upper section of FIG. 2 and the state illustrated in the lower section, depending on the process. In other words, the cartridge 200 is in the state illustrated in the upper section of FIG. 2 (in the state ejected from the incubator 2) in the process of feeding a cell fluid or a reagent and switching flow paths, and in the state illustrated in the lower section of FIG. 2 (in the state disposed inside the incubator 2) in the process of carrying out culture.

Although not illustrated in the figure, the cartridge 200 has a supply port for supplying the atmosphere in the interior space 22 to the spaces 230.

The configuration of the culture apparatus 1000 has been described above. In such a configuration, the culture apparatus 1000 enables an appropriate environment to be maintained in the culture chamber. As described above, a method that supplies water vapor into a culture chamber by a supply device provided outside the culture chamber is known as a means for controlling the environment in the culture chamber. However, in this method, water vapor is sometimes introduced into the culture chamber insufficiently, because the introduction of water vapor into the culture chamber is based on diffusion.

The culture apparatus 1000 according to the present embodiment therefore discharges the gas in the culture chamber to the outside to create a negative pressure in the culture chamber, so that gas is actively introduced into the culture chamber to make it possible to maintain an appropriate environment in the culture chamber. The details of the culture apparatus 1000 will now be described.

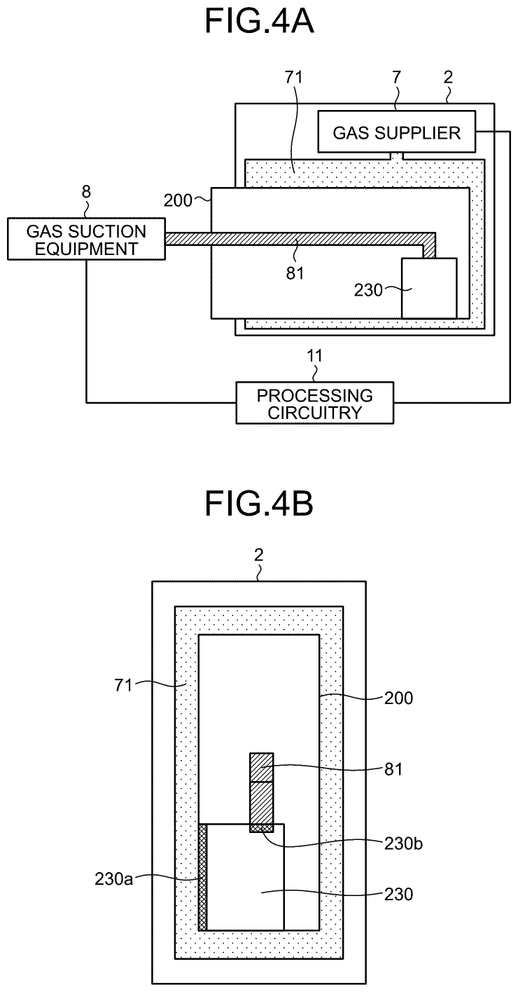

FIG. 4A and FIG. 4B are schematic diagrams illustrating an exemplary configuration of the culture apparatus 1000 according to the first embodiment. FIG. 4A schematically illustrates the culture apparatus 1000 viewed from a direction orthogonal to the direction of movement of the cartridge 200 by the transfer mechanism 6. FIG. 4B schematically illustrates the culture apparatus 1000 viewed from the cartridge 200 side in the state in which the cartridge 200 is inserted into the incubator 2.

As illustrated in FIG. 4A, the culture apparatus 1000 has a gas supplier 7, a gas suction equipment 8, a first flow path 71, and a second flow path 81, and maintains an appropriate environment in the space 230 (culture chamber) under the control of processing circuitry 11 contained in the apparatus base 1.

The gas supplier 7 is installed in the incubator 2 and supplies gas to the interior space 22 under the control of the processing circuitry 11. Specifically, the gas supplier 7 supplies various gases (CO₂, O₂, or N₂), water vapor, and the like so that the environment in the interior space 22 attains a set condition. For example, the gas supplier 7 supplies humidified gas to the interior space 22 so that the environment in the interior space 22 attains “humidity: 90%, and CO₂ concentration: 5%”.

Here, as illustrated in FIG. 4A, a space not occupied by the cartridge 200 in the interior space 22 having the cartridge 200 inserted serves as the first flow path 71. Thus, the gas supplier 7 supplies gas to the first flow path 71 formed to surround the cartridge 200. In this configuration, when the atmosphere in the interior space 22 is controlled to a condition suitable for cell culture (e.g., “temperature: 37° C., humidity: 90%, and CO2 concentration: 5%”), the atmosphere circulates around the cartridge 200 through the first flow path 71 to achieve the effect of heating the cartridge 200 and the space 230.

The first flow path 71 allows the gas supplied by the gas supplier 7 to flow to the culture chamber (space 230) for culturing a culture. As described above, the cartridge 200 has a supply port for supplying the atmosphere in the interior space 22 to the space 230, and the gas that flows through the first flow path 71 is supplied to the space 230 through the supply port.

The second flow path 81 is a flow path that connects the space 230 and the gas suction equipment 8 and through which the gas suctioned from the inside of the space 230 flows. For example, the second flow path 81 is formed inside the cartridge 200. Here, the cartridge 200 has an outlet port for discharging the gas from the space 230, and the second flow path 81 is connected to the outlet port and to the gas suction equipment 8.

The gas suction equipment 8 allows the gas in the culture chamber (space 230) to be discharged to the outside through the second flow path 81 under the control of the processing circuitry 11. For example, the gas suction equipment 8 is a vacuum pump to discharge gas at a discharge rate of 0.1 L/min to 20 L/min. The gas suction equipment 8 may be built in the cartridge 200 or may be installed outside the cartridge 200.

The processing circuitry 11 controls the discharge of gas by the gas suction equipment 8 and the supply of gas by the gas supplier 7 to maintain an appropriate environment in the space 230 (culture chamber). For example, the processing circuitry 11 is implemented by a processor. Here, the processing circuitry 11 controls various processes in the culture apparatus 1000 in response to various operations, for example, via a not-illustrated input interface.

For example, the processing circuitry 11 controls the gas supplier 7 to supply gas to the first flow path 71 and controls the gas suction equipment 8 to suction gas through the second flow path 81. In this way, gas is suctioned through the second flow path 81, whereby the gas is discharged from the space 230 through the outlet port connected to the second flow path 81. The discharge of the gas from the space 230 results in a negative pressure in the space 230, so that gas flows into the space 230 from the first flow path 71 through the supply port. With the above configuration, the culture apparatus 1000 according to the present embodiment enables the gas supplied from the gas supplier 7 to be actively introduced into the space 230, thereby maintaining an appropriate environment in the space 230.

Here, the processing circuitry 11 can control the gas supplier 7 to supply gas in a supply amount corresponding to a discharge amount. In other words, the gas supplier 7 supplies gas to the first flow path 71 in a supply amount corresponding to a discharge amount of gas discharged by the gas suction equipment 8. For example, the gas supplier 7 supplies gas to the first flow path in the same supply amount as the discharge amount.

The processing circuitry 11 controls the supply of gas and the discharge of gas as described above and also performs control to maintain the environmental condition in the interior space 22 based on information acquired by various sensors. For example, the processing circuitry 11 controls the temperature controller, the gas supplier, and the like so that the temperature, humidity, and CO₂ concentration are maintained at the set condition.

Further, the culture apparatus 1000 according to the present embodiment can supply clean gas to the space 230 (culture chamber). Specifically, the cartridge 200 has a supply port for gas to the space 230 and an outlet port for gas from the space 230, and a filter is installed at least at the supply port for gas. For example, as illustrated in FIG. 4B, a filter 230a is installed at the supply port through which gas is supplied from the first flow path 71, and a filter 230b is installed at the outlet port through which gas is discharged to the second flow path 81. The filters 230a and 230b are, for example, high efficiency particulate air filters (HEPA filters). Although FIG. 4B illustrates an example in which filters are installed at both the supply port and the outlet port, a filter may be installed only at the supply port. When filters are installed at both the supply port and the outlet port, the space 230 (culture chamber) can be humidified in a more suitable state while the sterility in the space 230 (culture chamber) is maintained.

### Modification

In the foregoing embodiment, an example in which a part of the interior space 22 serves as the first flow path 71 and the second flow path 81 is formed in the cartridge 200 has been described. In a modification, an example in which a part of the interior space 22 serves as the second flow path 81 and the first flow path 71 is formed in the cartridge 200 will be described. In other words, the culture apparatus 1000 according to the modification supplies gas directly to the space 230 and suctions gas through the interior space 22.

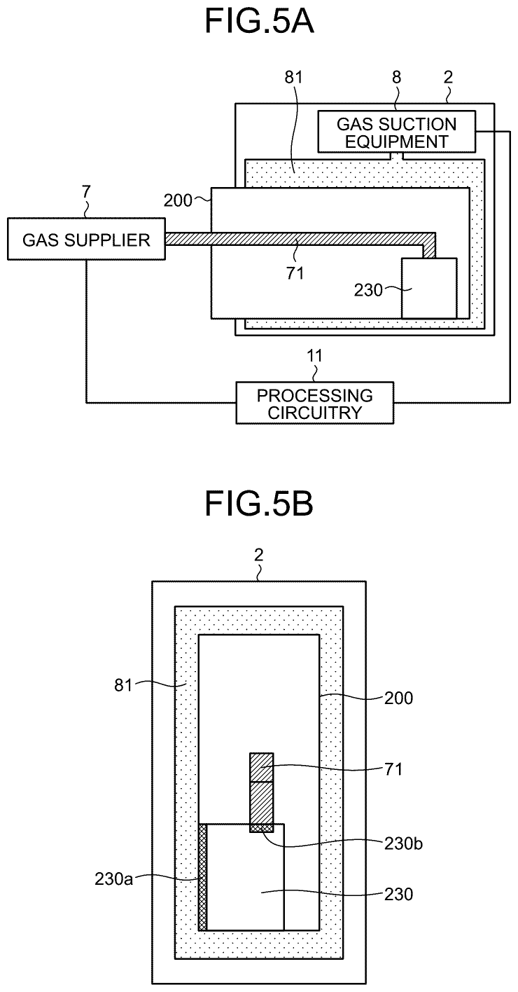

FIG. 5A and FIG. 5B are schematic diagrams illustrating an exemplary configuration of the culture apparatus 1000 according to the modification. FIG. 5A schematically illustrates the culture apparatus 1000 viewed from a direction orthogonal to the direction of movement of the cartridge 200 by the transfer mechanism 6. FIG. 5B schematically illustrates the culture apparatus 1000 viewed from the cartridge 200 side in the state in which the cartridge 200 is inserted into the incubator 2.

As illustrated in FIG. 5A, the gas supplier 7 according to the modification supplies gas to the first flow path 71 formed inside the cartridge 200.

As illustrated in FIG. 5A, the first flow path 71 according to the modification is formed inside the cartridge 200 and allows the gas supplied from the gas supplier 7 to flow directly into the space 230.

The second flow path 81 according to the modification is a space not occupied by the cartridge 200 in the interior space 22 having the cartridge 200 inserted and is formed to surround the cartridge 200 as illustrated in FIG. 5A and FIG. 5B.

The gas suction equipment 8 according to the modification is installed in the incubator 2 and suctions gas from the second flow path 81.

The processing circuitry 11 according to the modification controls the gas supplier 7 and the gas suction equipment 8 in the same manner as in the foregoing first embodiment. Here, in the culture apparatus 1000 according to the modification, the environment in the culture chamber can be maintained with a smaller amount of gas supply compared with the first embodiment, because gas can be supplied directly to the space 230.

In the culture apparatus 1000 according to the modification, as illustrated in FIG. 5B, filters (filter 230a and filter 230b) can also be installed at the supply port through which gas is supplied from the first flow path 71 and at the outlet port through which gas is discharged to the second flow path 81.

As described above, according to the first embodiment, the gas supplier 7 supplies gas. The space 230 (culture chamber) is configured to culture a culture. The first flow path 71 allows the gas supplied by the gas supplier 7 to flow to the space 230. The gas suction equipment 8 allows the gas in the space 230 to be discharged to the outside through the second flow path 81. The culture apparatus 1000 according to the first embodiment therefore enables an appropriate environment to be maintained in the culture chamber.

Further, according to the first embodiment, the gas supplier 7 supplies gas to the first flow path 71 in a supply amount corresponding to a discharge amount of gas discharged by the gas suction equipment 8. The culture apparatus 1000 according to the first embodiment therefore enables the supply amount of gas to be controlled as appropriate.

Further, according to the first embodiment, the space 230 (culture chamber) is provided in the cartridge 200 installed in the incubator 2 in which a culture environment is formed. The culture apparatus 1000 according to the first embodiment therefore enables an appropriate environment to be maintained in the culture chamber in the culturing using the cartridge 200.

Further, according to the first embodiment, the cartridge 200 has a supply port for gas to the space 230 (culture chamber) and an outlet port for gas from the culture chamber, and a filter is installed at least at the supply port for gas. The culture apparatus 1000 according to the first embodiment therefore enables clean air to be supplied to the culture chamber.

Further, according to the first embodiment, the first flow path 71 is formed to surround the cartridge 200. The culture apparatus 1000 according to the first embodiment therefore enables gas to be easily supplied to the culture chamber formed in the cartridge 200.

Further, according to the first embodiment, the gas discharge rate is 0.1 L/min to 20 L/min. The culture apparatus 1000 according to the first embodiment therefore enables adaptation to various culture environments.

Further, according to the modification, the second flow path 81 is formed to surround the cartridge 200. The culture apparatus 1000 according to the modification therefore enables gas to be easily discharged.

### Other Embodiments

In the foregoing embodiments, the culture chamber is formed in the cartridge 200 that allows for various processes related to cell culture. However, embodiments are not limited to this. For example, the culture chamber alone may be disposed in the incubator 2.

Further, in the foregoing embodiments, the culture is cells. However, embodiments are not limited to this. For example, the culture may be microorganisms.

The term “processor” as used herein refers to, for example, a central processing unit (CPU), a graphics processing unit (GPU), or circuitry such as an application-specific integrated circuit (ASIC) or a programmable logic device (e.g., simple programmable logic device (SPLD), complex programmable logic device (CPLD), and field programmable gate array (FPGA)). The processor reads and executes a computer program stored in a memory to implement a function. Instead of storing the computer program in the memory, the computer program may be directly embedded in circuitry of the processor. In this case, the processor reads and executes the computer program embedded in the circuitry to implement a function. Each processor in the present embodiment is not necessarily configured as a single circuit for each processor. A plurality of independent circuits may be combined as a single processor to implement its function.

Each constitutional element in each apparatus illustrated in connection with the description of the foregoing embodiments is a functional concept and does not necessarily have to be physically configured as illustrated in the figure. In other words, the specific form of distribution and integration of apparatuses is not limited to that illustrated in the figure. The apparatuses can be functionally or physically distributed or integrated in any units in their entirety or in parts according to loads, use conditions, or the like. Furthermore, the processing functions performed by the apparatuses can be implemented in their entirety or in parts by a CPU and a computer program analyzed and executed by the CPU, or implemented by hardware using wired logic.

As described above, according to the embodiments, an appropriate environment can be maintained in the culture chamber.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A culture apparatus comprising:
a gas supplier configured to supply gas;
a culture chamber configured to culture a culture;
a first flow path configured to allow gas supplied by the gas supplier to flow to the culture chamber; and
a gas suction equipment configured to allow gas in the culture chamber to be discharged to outside through a second flow path.

2. The culture apparatus according to claim 1, wherein the gas supplier supplies gas to the first flow path.

3. The culture apparatus according to claim 2, wherein the gas supplier supplies gas to the first flow path in a supply amount corresponding to a discharge amount of gas discharged by the gas suction equipment.

4. The culture apparatus according to claim 1, further comprising an enclosure in which a culture environment is formed; and a vessel installed in the enclosure, wherein the culture chamber is disposed in the vessel.

5. The culture apparatus according to claim 4, further comprising a filter, wherein the vessel has a supply port for gas to the culture chamber and an outlet port for gas from the culture chamber, and the filter is installed at least at the supply port for gas.

6. The culture apparatus according to claim 4, wherein the first flow path is formed to surround the vessel.

7. The culture apparatus according to claim 4, wherein the second flow path is formed to surround the vessel.

8. The culture apparatus according to claim 3, wherein the gas has a discharge rate of 0.1 L/min to 20 L/min.

9. A method comprising:
allowing gas supplied by a gas supplier to flow to a culture chamber through a first flow path, the culture chamber being configured to culture a culture; and
allowing gas in the culture chamber to be discharged by a gas suction equipment to outside through a second flow path.

10. The method according to claim 9, wherein gas is supplied to the first flow path by the gas supplier.

11. The method according to claim 10, wherein gas is supplied to the first flow path by the gas supplier in a supply amount corresponding to a discharge amount of gas discharged by the gas suction equipment.

12. The method according to claim 9, wherein the culture chamber is disposed in a vessel installed in an enclosure in which a culture environment is formed.

13. The method according to claim 12, wherein the vessel has a supply port for gas to the culture chamber and an outlet port for gas from the culture chamber, and a filter is installed at least at the supply port for gas.

14. The method according to claim 12, wherein the first flow path is formed to surround the vessel.

15. The method according to claim 12, wherein the second flow path is formed to surround the vessel.

16. The method according to claim 11, wherein the gas has a discharge rate of 0.1 L/min to 20 L/min.
